SEQUENCE LISTING (20 sequences)

SEQ ID NO: 1 - protein, 21 aa, Artificial sequence
  Synthetic oligopeptide
  MISC_FEATURE (13)..(13): Xaa is Ser/Ala
NKEIFLRELISNXSDALDKIR

SEQ ID NO: 2 - protein, 9 aa, Artificial sequence
  Synthetic oligopeptide
  MISC_FEATURE (6)..(6): Xaa is Lys/Arg
LGTIAXSGT

SEQ ID NO: 3 - protein, 16 aa, Artificial sequence
  Synthetic oligopeptide
  MISC_FEATURE (12)..(12): Xaa is Tyr/Phe
  MISC_FEATURE (16)..(16): Xaa is Glu/Asp
IGQFGVGFYSAXLVAX

SEQ ID NO: 4 - protein, 10 aa, Artificial sequence
  Synthetic oligopeptide
IKLYVRRVFI

SEQ ID NO: 5 - protein, 15 aa, Artificial sequence
  Synthetic oligopeptide
  MISC_FEATURE (6)..(6): Xaa is Glu/Asp
  MISC_FEATURE (12)..(12): Xaa is Ile/Val
GVVDSXDLPLNXSRE

SEQ ID NO: 6 - protein, 153 aa, Saccharomyces cerevisiae
MVVLNPNNWHWVDKNTLPWSKDYLNGKLTSLSTVSSDGKSKIELTQVSSITGDSNVSQRKGKPICYFDLQLSMNVKVTNLDTNKDDEDDDGILADGKLEIPEFMHDESDIPILSQGFDAFDGLVRSEFVPKVVETLLKYQDDLIKEHSKDIQV

SEQ ID NO: 7 - protein, 151 aa, Candida albicans
MVVHNPNNWHWIDKNCLPWSKDYLKENIIDTTYEDDSFRFVVTAVDSVSGDCDVTQRKGKVLCIYDMRLQFSLSGAIKKGNEEEEETISATIVIPEFVHDQDKDEYVFEIESASQKSEIRKYFVPILKEKLMRFQPDLLEAHAKDVQHATD

SEQ ID NO: 8 - protein, 350 aa, Saccharomyces cerevisiae
MVVNNPNNWHWVDKNCIGWAKEYFKQKLVGVEAGSVKDKKYAKIKSVSSIEGDCEVNQRKGKVISLFDLKITVLIEGHVDSKDGSALPFEGSINVPEVAFDSEASSYQFDISIFKETSELSEAKPLIRSELLPKLRQIFQQFGKDLLATHGNDIQVPESQVKSNYTRGNQKSSFTEIKDSASKPKKNALPSSTSTSAPVSSTNKVPQNGSGNSTSIYLEPTFNVPSSELYETFLDKQRILAWTRSAQFFNSGPKLETKEKFELFGGNVISELVSCEKDKKLVFHWKLKDWSAPFNSTIEMTFHESQEFHETKLQVKWTGIPVGEEDRVRANFEEYYVRSIKLTFGFGAVL

SEQ ID NO: 9 - protein, 360 aa, Arabidopsis thaliana
MAKFGEGDKRWIVEDRPDGTNVHNWHWSETNCLEWSRNFFTKQFSGVDILSGEGNLFIKVSKIEKVEGEAYVNVRKGKIIPGYELNVSLSWEGEAKDSDGKTLLKADGLVDMPYISDENADEDPEIRFSVKDEGPIGRTLKEAMVKKGKEIILEKVRVYVEAMARGGPCRDELESKKVAPKSVAAGSATVAVEKSGAAPVVSAAAVESKVVKEKKKAKTKEGFKTITMTEKFNCRARDLYEILMDENRWKGFTQSNAKISKDVNGPISVFDGSVTGMNLELEEGKLIVQKWRFGSWPDGLDSTVKIVFEEPQPGVTIVNLTHTDVPEEDRYGNATVVENTERGWRDLIFHRIRAVFGFGI

SEQ ID NO: 10 - protein, 361 aa, Drosophila melanogaster
MAKWGEGDPRWIVEERPDATNVNNWHWTEKNATPWSKDRLHQLFQDFKIGEYIRICRQSDIECAVDSVDKCSGEATVNNRKGKLIFFYEWELVLKWSGKLLKNSKLIHKGKLTIPNLSEENELADVEITVTIDESNDESETLKQFMYNVGRDRVRQQLASYIRELKEEYSKNLILPKKGDEAGAGNTVANFKDANNTRNAAQNIALNSSVAAPRLKNSGIGCKLDVRTLSMTEEFHCSANDLYNALTKPEMVTAFTRAPAKVDAVRGGEFILYGGNVLGKFEELVPEKKIQQSWRLKNWTSGHYSNVVIELEETSSSTMMSLKQTGIPASEFDAMKTNWYRYYWHSIKQTFGFGTSISDAL

SEQ ID NO: 11 - protein, 342 aa, Caenorhabditis elegans
MAKWGEGDPRWIVEERADATNVNNWHWSEKNATPWSLNRLRELLTGFSSEDGPIVVTIDEIKKIDGEATANNRKAKLIFLFEWVIEGTFIARVSGSEDEYKGTFDIPNLSDENDASEVDVNSSLSGNGPMAHQIRQVLNKSFIAKIQDVMGIYIRELKEEFSKGLILPTDKVKPQVVTTGKTSVVDKRQFQNTVVAEKDASTSGNEVFSTKEVSTSDTYKATPDRVFEALTETQFVRGWTNNSIGEWNFKEGGSFALFGENVTGTFEKIEPNKEIVKKWRLKKYPNNHHATIHFQLKDTGSGTDIKIIAKDVPTHLAEETQQGLDRYYLSSIGRTFGFSQRM

SEQ ID NO: 12 - protein, 338 aa, Homo sapiens
MAKWGEGDPRWIVEERADATNVNNWHWTERDASNWSTDKLKTLFLAVQVQNEEGKCDVTEVSKLDGEASINNRKGKLIFFYEWSVKLNWTGTSKSGVQYKGHVEIPNLSDENSVDEVEISVSLAKDEPDTNLVALMKEEGVKLLREAMGIYISTLKTEFTQGMILPTMNGESVDPVGQPALKTEERKAKPAPSKTQARPVGVKIPTCKITLKETFLTSPEELYRVFTTQELVQAFTHAPATLEADRGGKFHMVDGNVSGEFTDLVPEKHIVMKWRFKSWPEGHFATITLTFIDKNGETELCMEGRGIPAPEEERTRQGWQRYYFEGIKQTFGYGARLF

SEQ ID NO: 13 - protein, 336 aa, Schizosaccharomyces pombe
MSATSINPNNWHWTSKDCRVWSHEYFNKELPKIQASEGPTSARITQVNSCEGDVDVSMRKRKVITIFDLKIQMEFKGETKDGVEATGSITCPELSYDLGYSDYVFDIDIYSASKEKEPIKELVREKIIPQIRQLFSGFSQVLLQTHGDDVYLSTEEHNGNAARGLPVHSSFKQNNSSQTSSNKGTTTVAAGSGSDGSRVSAVVNTADISENYTFDAPANELYATFLDPARVAAWSRAPPQLDVRPQGAFSLFHGNVVGKFLVLEENKKIVQTWRLSSWPTGHYAEITFTFDQADSYTTLRMIMKGVPIGEEEVVQGNIQDYYIRPIKTVFGFGAVL

SEQ ID NO: 14 - protein, 140 aa, Plasmodium falciparum
MSFEITEEYYVPPEVLFNAFTDAYTLTRLSRGSLAEVDLKVGGKFSLFSGSILGEFTEITKPHKIVEKWKFRDWNECDYSTVTVEFISVKENHTKLKLTHNNIPASNKYNEGGVLERCKNGWTQNFLHNIEVILGYPKKK

SEQ ID NO: 15 - protein, 20 aa, Artificial sequence
  Synthetic oligopeptide
  MISC_FEATURE (2)..(2): Xaa is any amino acid
  MISC_FEATURE (8)..(10): Xaa is any amino acid
  MISC_FEATURE (12)..(14): Xaa is any amino acid
  MISC_FEATURE (17)..(17): Xaa is any amino acid
  MISC_FEATURE (19)..(19): Xaa is any amino acid
  MISC_FEATURE (3)..(3): Xaa may be Asn/His
  MISC_FEATURE (11)..(11): Xaa may be Asp/Asn
  MISC_FEATURE (16)..(16): Xaa may be Ser/Ala
  MISC_FEATURE (18)..(18): Xaa may be Asp/Asn/Glu
  MISC_FEATURE (20)..(20): Xaa may be Phe/Leu
NXXNWHWXXXXXXXWXXXXX

SEQ ID NO: 16 - protein, 18 aa, Artificial sequence
  Synthetic oligopeptide
  MISC_FEATURE (3)..(4): Xaa is any amino acid
  MISC_FEATURE (6)..(7): Xaa is any amino acid
  MISC_FEATURE (10)..(10): Xaa is any amino acid
  MISC_FEATURE (14)..(14): Xaa is any amino acid
  MISC_FEATURE (2)..(2): Xaa is Asp/Glu
  MISC_FEATURE (5)..(5): Xaa may be Val/Ala/Ile
  MISC_FEATURE (12)..(12): Xaa may be Pro/Val/Ile/Leu
  MISC_FEATURE (13)..(13): Xaa is Ile/Leu
  MISC_FEATURE (15)..(15): Xaa is Phe/Tyr
  MISC_FEATURE (16)..(16): Xaa is Asp/Glu
  MISC_FEATURE (17)..(17): Xaa may be Leu/Met/Trp
  MISC_FEATURE (18)..(18): Xaa may be Gln/Arg/Lys/Asn/Glu/Ser/Val
GXXXXXXRKXKXXXXXXX

SEQ ID NO: 17 - protein, 5 aa, Artificial sequence
  Synthetic oligopeptide
  MISC_FEATURE (2)..(2): Xaa is Arg/Lys
  MISC_FEATURE (3)..(3): Xaa is Phe/Leu
  MISC_FEATURE (5)..(5): Xaa is Trp/Tyr
  MISC_FEATURE (4)..(4): Xaa is any amino acid
WXXXX

SEQ ID NO: 18 - protein, 11 aa, Artificial sequence
  Synthetic oligopeptide
  MISC_FEATURE (3)..(4): Xaa is any amino acid
  MISC_FEATURE (6)..(8): Xaa is any amino acid
  MISC_FEATURE (1)..(1): Xaa may be Tyr/Ile/Phe
  MISC_FEATURE (2)..(2): Xaa may be Val/Phe/Trp/Ile/Leu
  MISC_FEATURE (9)..(9): Xaa is Phe/Leu
  MISC_FEATURE (11)..(11): Xaa is Phe/Tyr
XXXXIXXXXGX

SEQ ID NO: 19 - protein, 6 aa, Saccharomyces cerevisiae
MVVNNP

SEQ ID NO: 20 - protein, 6 aa, Saccharomyces cerevisiae
GNDIQV

(19) United States
(12) Patent Application Publication
(10) Pub. No.: US 2007/0010432 A1
(43) Pub. Date: Jan. 11, 2007

(54) Heat shock protein 90 activator

(76) Inventors: Paul Workman, SUTTON, SURREY (GB); Wynne Ahern, Sutton, Surrey (GB); Laurence Pearl, London, Greater London (GB); Chrisostomos Prodromou, London, Greater London (GB)

Correspondence Address: KLARQUIST SPARKMAN, LLP, 121 SW SALMON STREET, SUITE 1600, PORTLAND, OR, 97204, US

(21) Appl. No.: 10/503,867

(22) PCT Filed: Feb. 4, 2003
(86) PCT No.: PCT/GB03/00492

§ 371 Date: Mar. 31, 2005

(30) Foreign Application Priority Data
Feb. 7, 2002 (GB) 0202871.0

(51) Int. Cl. A61K 38/54; G01N 33/567; G01N 33/53

(52) U.S. Cl. 514/12; 435/7.2

(57) ABSTRACT

This invention relates the identification of a novel co-factor (termed ‘Aha1’) that interacts with the molecular chaperone Heat shock protein 90 (Hsp90) and stimulates Hsp90 activity. Various assay methods and therapeutic applications based on this interaction are provided.

The present invention relates to the identification of an interaction between the molecular chaperone Heat shock protein 90 (Hsp90) and the co-factor, Aha1. This interaction stimulates the activity of Hsp90 and allows the development of assay and other methods based on the modulation of Hsp90 activity.

Heat shock protein 90 (Hsp90) constitutes about 1-2% of total cellular protein, and is usually present in the cell as a dimer in association with one of a number of other proteins (see, e.g., Pratt, W. B. (1997) Annu. Rev. Pharmacol. Toxicol. Vol. 37, pp. 297-326). Hsp90 is essential for cell viability and exhibits dual chaperone functions (Young J. C. et al (2001) J. Cell. Biol., Vol. 154, pp. 267-273). It plays a key role in the cellular stress response by interacting with many proteins after their native conformation has been altered by environmental stresses, such as heat shock, ensuring adequate protein folding and preventing non-specific aggregation (Smith et al., (1998) Pharmacological Review, Vol. 50, pp; 493-513). In addition, recent evidence suggests that Hsp90 may also play a role in buffering against the effects of mutation, presumably by correcting the inappropriate folding of mutant proteins.

Hsp90 also has an important regulatory role. Under normal physiological conditions, together with its endoplasmic reticulum homologue GRP94 (acc no: P14625), Hsp90 plays a housekeeping role in the cell, maintaining the conformational stability and maturation of several key client proteins. These can be sub-divided into three groups: (a) steroid hormone receptors, (b) Ser/Thr or tyrosine kinases (e.g., ErbB2, RAF-1, CDK4, and LCK), and (c) a collection of apparently unrelated proteins, e.g. mutant p53 and the catalytic subunit of telomerase hTERT. These proteins play key regulatory roles in many physiological and biochemical processes in the cell.

Because it is involved in regulating a number of signalling pathways that are crucially important in driving the phenotype of a tumour, the molecular chaperone HSP90 represents a potentially valuable target for anti-cancer drug development.

Although an inherent ATPase activity has been shown for yeast Hsp90 (Panaretou B et al (1998) supra), E. coli Hsp90 (HtpG) and the mammalian homologue Trap1 (Felts et al (2000) J. Biol. Chem. 275 3305-3312: AAC02679), a geldanamycin-sensitive ATPase activity has proved to be difficult to demonstrate in other Hsp90 polypeptides, including human Hsp90.

The present inventors have unexpectedly discovered that the previously uncharacterised Aha1 protein stimulates the ATPase activity of Hsp90. This stimulation allows the ATPase activity of human Hsp90 to be detected for the first time. The stimulation of Hsp90 geldanamycin-sensitive ATPase activity by Aha1 allows screening assays to be performed to identify compounds which modulate (i.e. increase or decrease) Hsp90 activity, for example by interfering with the interaction between Aha1 and Hsp90.

Based on the experimental work and discussion herein, the present invention is concerned in various aspects with the interaction of Hsp90 and Aha1, the stimulation of Hsp90 activity by Aha1 and the modulation of this Aha1-mediated Hsp90 activity.

Various aspects of the present invention provide for the use of an Aha1 polypeptide in screening methods and assays for identifying and/or obtaining agents that modulate the activity of Hsp90, in particular, the ATPase activity.

One general aspect of the invention provides a method for identifying and/or obtaining an agent which modulates the activity of an Aha1 polypeptide, including;
(a) bringing into contact an Aha1 polypeptide and a test compound; and,
(b) determining binding between said Aha1 polypeptide and said test compound.

The presence of binding is indicative of the test compound being a putative agent that modulates Aha1 activity. A suitable Aha1 activity may include the stimulation of Hsp90 ATPase activity.

A method for identifying and/or obtaining an agent which modulates the interaction between Aha1 and Hsp90 may include;
(a) bringing into contact an Hsp90 polypeptide, a Aha1 polypeptide and a test compound; and,
(b) determining interaction between said Aha1 polypeptide and said Hsp90 polypeptide.

Interaction of the Aha1 polypeptide with Hsp90 polypeptide in the presence of a test compound may be compared with the interaction of the Aha1 polypeptide to Hsp90 polypeptide in comparable reaction medium and conditions in the absence of a test compound.

A difference (i.e. an increase or decrease) in interaction in the presence of test compound relative to the absence is indicative that the test compound is an agent which modulates the interaction of Aha1 and Hsp90.

Test compounds which reduce or inhibit the interaction of an Aha1 polypeptide and an Hsp90 polypeptide may be identified using conditions which, in the absence of a positively-testing agent, allow such interaction to occur. Such compounds may be used as agents to inhibit the function of Hsp90, for example in the treatment of Hsp90-mediated disorders, and may have an effect, for example, on cellular activities such as proliferation as described below.

Test compounds which increase or enhance the interaction of an Aha1 polypeptide and an Hsp90 polypeptide may be identified using conditions which, in the absence of a positively-testing agent, prevent such interaction occurring. Such compounds may be used as agents to potentiate the function of Hsp90, for example in the treatment of Hsp90 mediated conditions, and may have an effect for example on cellular functions such as proliferation.

Determining the interaction between an Aha1 polypeptide and an Hsp90 polypeptide may include determining the binding between an Aha1 polypeptide and an Hsp90 polypeptide and/or determining stimulation or enhancement of Hsp90 activity, for example Hsp90 ATPase activity, in the presence of an Aha1 polypeptide.

An Hsp90 polypeptide suitable for use in accordance with the present invention may be a eukaryotic Hsp90, preferably a mammalian Hsp90, or a mutant, homologue, variant, derivative or allele thereof. A suitable polypeptide may have a sequence of an Hsp90 protein as shown in Table 1 or may be a variant, allele, derivative or mutant thereof. In some preferred embodiments, the Hsp90 polypeptide is a human Hsp90 polypeptide, and may for example be any one of the human isoforms HSP90α, Hsp90β, GRP94 or HSP75/TRAP1, for example HSP90α, Hsp90β or GRP94.

A Hsp90 polypeptide which is a variant, allele, derivative or mutant of a Hsp90 polypeptide shown in Table 1 may comprise an amino acid sequence which shares greater than about 50% sequence identity with the sequence of a Hsp90 polypeptide shown in Table 1, greater than 55%, greater than about 60%, greater than about 65%, greater than about 70%, greater than about 75%, greater than about 80%, greater than about 85%, greater than about 90%, greater than about 95% or greater than about 98%. Such a variant, allele, derivative or mutant of a Hsp90 polypeptide of Table 1 preferably has the Hsp90 activity of a Hsp90 polypeptide of Table 1.

Preferably, an Hsp90 polypeptide for use in methods of the present invention comprises one or more of the following sequence motifs:

(a) NKEIFLRELISN(S/A)SDALDKIR
(b) LGTIA(K/R)SGT
(c) IGQFGVGFYSA(Y/F)LVA(E/D)
(d) IKLYVRRVFI
(e) GVVDS(E/D)DLPLN(I/V)SRE

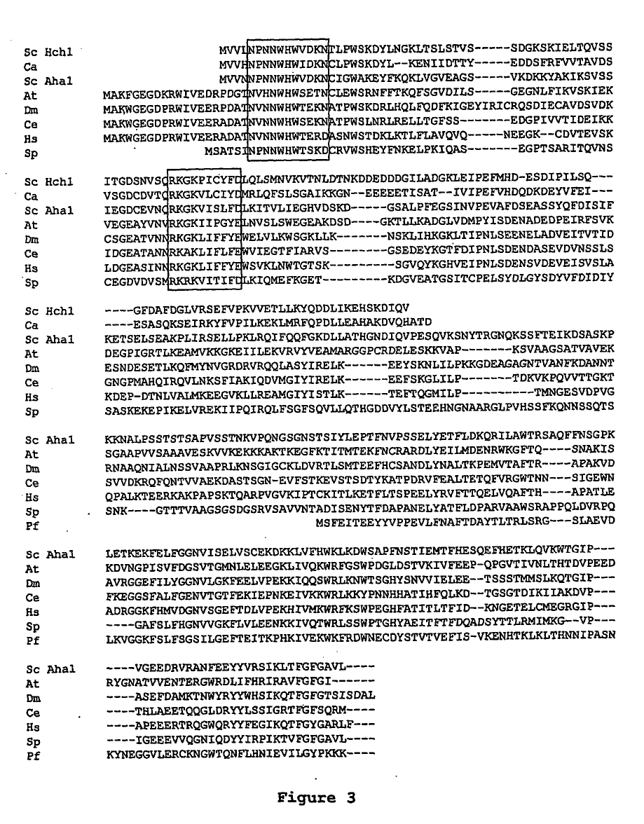

An Aha1 polypeptide suitable for use in accordance with the present invention may be a eukaryotic Aha1. Suitable polypeptides may have a sequence of an Aha1 protein as shown in FIG. 3 or may have a sequence which differs from the sequences shown in FIG. 3 but which is a variant, allele, derivative or mutant thereof. In some preferred embodiments, the Aha1 polypeptide may be a yeast (S. cervisiae) polypeptide encoded by a nucleic acid having the sequence of database accession number YDR214W (Saccharomyces genome database no: S0002622) or a human polypeptide (database accession no: AJ243310).

A variant, allele, derivative or mutant may comprise an amino acid sequence which shares greater than about 35% sequence identity with the sequence of yeast Aha1 (Database accession number: YDR214W) or other sequence shown in FIG. 3 such as Hch1, greater than 55%, greater than about 60%, greater than about 65%, greater than about 70%, greater than about 75%, greater than about 80%, greater than about 85%, greater than about 90%, greater than about 95% or greater than about 98%.

Particular amino acid sequence variants may differ from a known Hsp90 or Aha1 polypeptide sequence as described herein by insertion, addition, substitution or deletion of 1 amino acid, 2, 3, 4, 5-10, 10-20 20-30, 30-50, or more than 50 amino acids.

Preferably, an Aha1 polypeptide may comprise one or more of the following sequence motifs:

(a) NX*¹NWHWXXX(D/N)XXXW(S/A)X*²X(F/L)
(b) G(D/E)XX*³XXRKXK*⁴(I/L)X(F/Y)(D/E)*⁵*⁶
(c) W(R/K)(F/L)X(W/Y)
(d) *⁷*⁸XXIXXX(F/L)G(F/Y)

where X is any amino acid and * is a position with a number of conservative changes. For example, *¹ may be (N/H), *² may be (D/N/E), *³ may be (V/A/I), *⁴ may be (P/V/I/L), *⁵ may be (L/M/W), *⁶ may be (Q/R/K/N/E/S/V), *⁷ may be (Y/I/F) and *⁸ may be (V/F/W/I/L).

An Hsp90 or Aha1 polypeptide may consist of a portion, segment or fragment of a full-length polypeptide sequence, for example a sequence of Table 1 or FIG. 3, which retains the activity of the full length Hsp90 or Aha1 polypeptide, in particular, such a fragment binds to a Aha1 or Hsp90 polypeptide, respectively.

In preferred embodiments, an Aha1 polypeptide which is a fragment of the full-length polypeptide may comprise the N terminal domain, as described herein.

A “fragment”, “portion” or “segment” of a polypeptide generally means a stretch of amino acid residues which is shorter than the full length amino acid sequence, for example less than 411 amino acids, less than 400 amino acids, less than 350 amino acids, less than 300 amino acids, less than 250 amino acids, less than 200 amino acids, less than 150 amino acids, less than 100 amino acids, less than 50 amino acids, less than 30 amino acids or less than 25 amino acids. A fragment will generally consist of at least 5 amino acids, for example at least 7 amino acids, at least 10 amino acids, at least 15 amino acids, at least 20 amino acids, at least 25 amino acids, at least 30 amino acids or at least 35 amino acids.

An Aha1 polypeptide which stimulates the geldanamycin sensitive ATPase activity of Hsp90 as described above is provided as an aspect of the present invention.

Sequence identity is commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4.

Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol. Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Sequence comparison may be made over the full-length of the relevant sequence described herein, or may more preferably be over a contiguous sequence of about or greater than about 20, 25, 30, 33, 40, 50, 67, 133, 167, 200, 233, 267, 300, 333, or more amino acids or nucleotide triplets, compared with the relevant amino acid sequence or nucleotide sequence as the case may be.

Interaction between two proteins, as described herein, means that the two proteins bind specifically to each other through non-covalent intermolecular bonds.

The determination of interaction or activity may be quantitative or qualitative and may include detecting the existence of the interaction or activity, which may, for example, include detecting the existence of an interaction or activity above a certain threshold value, and measuring the amount or level of the interaction or activity.

Another aspect of the present invention provides a method for identifying and/or obtaining an agent which modulates ATPase activity of Hsp90, the method comprising:
(a) bringing into contact an Hsp90 polypeptide, an Aha1 polypeptide and a test compound; and,
(b) determining ATPase activity of said Hsp90 polypeptide. Hsp90 ATPase activity may be geldanamycin-sensitive ATPase activity. Suitable Hsp90 and Aha1 polypeptides are described in more detail above.

The Hsp90 polypeptide, Aha1 polypeptide and test compound may be brought into contact under conditions in which, in the absence of the test compound, ATPase activity of Hsp90 is observed.

The ATPase activity of the Hsp90 polypeptide in the presence of a test compound may be compared with the ATPase activity of the Hsp90 polypeptide in comparable reaction medium and conditions in the absence of a test compound.

A change (i.e. an increase or a decrease) in the Hsp90 ATPase activity in the presence of test compound relative to the absence is indicative that the test compound is an agent which modulates Hsp90 activity.

ATPase activity may be determined using standard assays as described herein, for example, by determining the production of inorganic phosphate (Pᵢ). Pᵢ production may be determined, for example, by measuring or determining the generation or depletion of a reporter molecule. In one suitable format described below, a detectable reporter molecule is produced by the reaction of a cationic malachite green dye with a phosphomolybdate complex.

ATPase activity may be determined in the presence and absence of test compound. A change (i.e. a difference such as an increase or decrease) in said activity in the presence relative to the absence of test compound being indicative of said test compound being an agent which modulates ATPase activity of Hsp90. A decrease in activity is indicative of the test compound being an agent which inhibits the ATPase activity of Hsp90 and an increase in activity is indicative of the test compound being an agent which stimulates the ATPase activity of Hsp90. Methods for determining ATPase activity in a test sample may include quantifying the amount of substrate in the sample.

The test compound may be brought into contact with the Hsp90 polypeptide and Aha1 polypeptide in the presence of a suitable substrate such as ATP.

Hsp90 ATPase activity may be determined in the presence and absence of geldanamycin. A change in ATPase activity (e.g. a decrease, abrogation or abolition of ATPase activity) in the presence, relative to the absence, of geldanamycin is indicative of the ATPase activity being geldanamycin sensitive.

Methods of the invention may include identifying said test compound as an agent which modulates the activity of Hsp90.

Methods of the invention may be used to determine the amount or level of Hsp90 in a test sample. In such an assay, the ATPase activity of Hsp90 in a sample may be determined and compared to a calibration curve to determine the amount or level of Hsp90 present in the sample. A suitable calibration curve may be produced by determining the activity of known amounts of protein (i.e. protein standards), for example, using recombinant/expressed protein of known purity.

It is not necessary to use entire proteins for methods of the invention which test for binding between two molecules. Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system; Another recombinant approach is to amplify the relevant portion of the DNA with suitable PCR primers. Small fragments (e.g. up to about 20 or 30 amino acids) may also be generated using peptide synthesis methods which are well known in the art.

Suitable fragments of Hsp90 as described above are those which retain the ATPase activity of the full-length polypeptide. Suitable fragments of Aha1 are those which retain the ability to enhance Hsp90 ATPase activity, for example fragments comprising the Aha1 N terminal domain. Smaller fragments, and analogues and variants of these fragments may similarly be employed, e.g. as identified using techniques such as deletion analysis or alanine scanning. Residues which are conserved between species and play a role in Aha1 activity are shown in FIG. 3.

A test compound may be a small chemical entity, peptide, antibody molecule or other molecule whose effect on the interaction between Aha1 and Hsp90 or the Aha1 mediated ATPase activity of Hsp90 is to be determined. Suitable test compounds may be selected from compound collections and designed compounds, for example using combinatorial chemistry as described below. Methods of the invention may be used to determine whether a test compound is an agent which modulates Hsp90 ATPase activity.

Those of skill in the art may vary the precise format of any of the screening or assay methods of the invention using routine skill and knowledge. The skilled person is well-aware of the need to employ appropriate control experiments.

Methods of determining the interaction of Aha1 and Hsp90 and of screening for an agent able to modulate the ATPase activity of Hsp90 include methods in which a suitable endpoint is used to assess interaction. Interaction may be determined by any number of techniques known in the art, qualitative or quantitative. They include techniques such as radioimmunoassay, co-immunoprecipitation, scintillation proximity assay, ELISA, Fluorescence resonance energy transfer (FRET), gene expression micro-arrays and Northern blot methods.

Other suitable end points include measuring the effect on Rsp90 ATPase activity by the determination of changes in the levels of ATP, ADP or Pᵢ using conventional methods as described in more detail below.

Binding of Aha1 to a binding partner such as Hsp90 may be studied by labelling either one with a detectable label and bringing it into contact with the other, which may have been immobilised on a solid support.

Suitable detectable labels, especially for peptidyl substances, include ³⁵S-methionine, which may be incorporated into recombinantly produced peptides and polypeptides. Recombinantly produced peptides and polypeptides may also be expressed as fusion proteins containing an epitope which can be labelled with an antibody.

The peptide which is immobilized on a solid support may be immobilized using an antibody against that peptide bound to a solid support or via other technologies which are known per se, for example using the biotin/streptavidin interaction. A preferred in vitro interaction may utilise a fusion peptide including glutathione-S-transferase (GST). This may be immobilized on glutathione agarose beads. In an in vitro assay format of the type described above a test modulator can be assayed by determining its ability to diminish the amount of labelled peptide (e.g. labelled Aha1 which binds to the immobilized GST-fusion peptide (e.g. immobilised fusion peptide of GST and a peptide comprising Hsp90). This may be determined by fractionating the glutathione-agarose beads by SDS-polyacrylamide gel electrophoresis. Alternatively, the beads may be rinsed to remove unbound peptide and the amount of peptide which has bound can be determined by counting the amount of label present in, for example, a suitable scintillation counter.

Binding or interaction of Aha1 with a binding partner such as Hsp90 may also be determined using a two-hybrid assay.

For example, Aha1 polypeptide, Hsp90 polypeptide or a fragment of either of these may be fused to a DNA binding domain such as that of the yeast transcription factor GAL4. The GAL4 transcription factor includes two functional domains. These domains are the DNA binding domain (GAL4DBD) and the GAL4 transcriptional activation domain (GAL4TAD). By fusing Aha1 polypeptide or a fragment thereof to one of those domains, and Hsp90 polypeptide or a fragment thereof to the respective counterpart, a functional GAL4 transcription factor is restored only when the two peptides interact. Thus, interaction of these peptides may be measured by the use of a reporter gene linked to a GAL4 DNA binding site which is capable of activating transcription of said reporter gene.

This two hybrid assay format is described by Fields and Song, 1989, Nature 340; 245-246. It can be used in both mammalian cells and in yeast. Other combinations of DNA binding domain and transcriptional activation domain are available in the art and may be preferred, such as the LexA DNA binding domain and the VP60 transcriptional activation domain.

When looking for substances which interfere with the interaction of Aha1 polypeptide with a Hsp90 polypeptide, and which may therefore modulate the Aha1 mediated ATPase activity of Hsp90, a peptide fragment of Aha1 or Hsp90 may be employed as a fusion with (e.g.) the LexA DNA binding domain, and the counterpart peptide fragment containing Hsp90 or Aha1 as a fusion with (e.g.) VP60. An expression cassette may be used to express a test peptide within a host cell.

As described above, methods of the present invention may comprise determining ATPase activity. Two methods have been described to measure the intrinsic ATPase activity of HSP90, both using yeast HSP90 as a model system.

The first method utilises a regenerating coupled enzyme assay. A regenerating ATPase assay may be performed using the pyruvate kinase/lactate dehydrogenase linked assay described by Ali et al, 1993 et al Biochemistry Vol. 32, pp. 2717-2724.

The ADP that is generated by HSP90 is phosphorylated by pyruvate kinase, utilising phosphoenol pyruvate (PEP) as substrate, to produce ATP and pyruvate as products. Pyruvate is then converted to lactic acid by lactate dehydrogenase utilising NADH, which is converted to NAD.

This consumption of NADH concentration leads to a decrease in the absorbance at 340 nm that is monitored spectrophotometrically. Thus, for every mole of ADP that is generated by the ATPase activity of HSP90, one mole of NADH is utilised. It should be noted that prior to the addition of HSP90, the enzyme system converts any contaminating ADP, which is present in the ATP substrate, to ATP. This is important for enzymes such as HSP90, which show a stronger affinity for the binding of ADP than ATP.

A second method, based on the use of malachite green for the measurement of inorganic phosphate, was designed for high throughput screening (HTS) to identify novel HSP90 inhibitor drug candidates. Colorimetric assays for the determination of phosphate, based on the formation of a phosphomolybdate complex, such as the malachite green ATPase assay, can be carried out in a few steps with inexpensive reagents and are well suited to the automation required for high throughput screening (see, e.g., Cogan et al. (1999) Anal. Biochem. Vol. 271, pp. 29-35).

Enzymes that release inorganic phosphate are assayed using the reaction of the cationic dye, malachite green, with the phosphomolybdate complex to generate a blue-green colour with an absorbance maximum at 610 nm (see, e.g., Baykov et al., (1988) Anal. Biochem., Vol. 171, pp. 266-270.; Harder et al., (1994) Biochem. J. Vol. 298, pp. 395-401.; Maehama et al., (2000) Anal. Biochem. Vol. 279, pp. 248-250).

The method has been used in both high throughput (see, e.g., Rumsfeld et al., (2000) Protein Expr. Purif. Vol. 18, pp. 303-309) and ultra-high throughput screening formats (see, e.g., Lavery et al., (2001) J. Biomol. Screen. Vol. 6, pp. 3-9). However, this method is complicated by the non-enzymatic hydrolysis of ATP in the presence of acidic malachite green reagent, causing an increase in colour (see, e.g., Chan et al., (1986) Anal. Biochem. Vol. 157, pp. 375-380; Henkel et al., 1988 Anal. Biochem. Vol. 169, pp. 312-318.). This process is mediated by molybdate and can be overcome by the addition of sodium citrate immediately after the reagent (see, e.g., Lanzetta et al. (1979) Anal. Biochem. Vol. 100, pp. 95-97; Schirmer et al.(1998) J. Biol. Chem. Vol. 273, pp. 15546-15552; Baginski et al., (1975) Ann. Clin. Lab. Sci. Vol. 5, pp. 399-416). This modification has been adapted to the 96-well microtitre plate assay previously described for other ATPases (see, e.g., Lanzetta et al. (1979) supra) to produce the following protocol for HSP90 ATPase, which is suitable for high throughput screening.

Compounds which may be screened may be natural or synthetic chemical compounds used in drug screening programmes. Extracts of plants, microbes or other organisms, which contain several characterised or uncharacterised components may also be used.

Combinatorial library technology is an example of an efficient method of testing a potentially vast number of different substances for ability to modulate an interaction. Such libraries and their use are known in the art, for all manner of natural products, small molecules and peptides, among others. The use of peptide libraries may be preferred in certain circumstances.

The amount of test substance or compound which may be added to a method of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM. Even a molecule which elicits a weak effect may be a useful lead compound for further investigation and development.

One class of putative inhibitor compounds can be derived from the Aha1 polypeptide and/or the Hsp90 polypeptide which interacts with it. Membrane permeable peptide fragments of from 5 to 40 amino acids, for example, from 6 to 10 amino acids may be tested for their ability to disrupt such interaction or activity.

Peptide fragments may be obtained by means of deletion analysis and/or alanine scanning of the Aha1 sequence—making an appropriate mutation in sequence, bringing together a mutated fragment of Aha1 with Hsp90 or a fragment thereof and determining interaction, preferably by measuring or monitoring the ATPase activity of Hsp90. In preferred embodiments, the peptide is short, as discussed below, and may be a minimal portion that is able to interact with Hsp90 and/or inhibit the relevant interaction. Similarly, peptide fragments of Hsp90 which are able to inhibit the interaction of Aha1 and Hsp90 may also be used to inhibit the Aha1 induced ATPase activity of Hsp90.

The skilled person can use the techniques described herein and others well known in the art to produce large amounts of peptides, for instance by expression from encoding nucleic acid.

Peptides can also be generated wholly or partly by chemical synthesis. The compounds of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, is liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

The inhibitory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Antibodies directed to the site of interaction in either the Hsp90 polypeptide or the Aha1 polypeptide, form a further class of putative inhibitor compounds. Candidate inhibitor antibodies may be characterised and their binding regions determined to provide single chain antibodies and fragments thereof which are responsible for disrupting the interaction.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed, the term “antibody” should be construed as covering any binding substance having a binding domain with the required specificity. Thus the invention covers antibody fragments, derivatives, functional equivalents and homologues of antibodies, including synthetic molecules and molecules whose shape mimicks that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. The mode of determining binding is not a feature of the present invention and those skilled in the art are able to choose a suitable mode according to their preference and general knowledge.

Antibodies may also be used in purifying and/or isolating a polypeptide or peptide for use in the present methods, for instance following production of the polypeptide or peptide by expression from encoding nucleic acid therefor.

Antibodies may be useful in a therapeutic context (which may include prophylaxis) to disrupt Hsp90 interactions co-factors such as Aha1 with a view to inhibiting Hsp90 activity. Antibodies can for instance be micro-injected into cells, e.g. at a tumour site, subject to radio- and/or chemo-therapy (as discussed already above). Antibodies may be employed in accordance with the present invention for other therapeutic and non-therapeutic purposes which are discussed elsewhere herein.

Other candidate inhibitor compounds may be based on modelling the 3-dimensional structure of a polypeptide or peptide fragment and using rational drug design to provide potential modulator (for example, inhibitor) compounds with particular molecular shape, size and charge characteristics.

A potential modulator compound may be a “functional analogue” of a peptide or other compound which modulates Aha1/Hsp90 binding or Hsp90 activity in a method of the invention. A functional analogue has the same functional activity as the peptide or other compound in question, i.e. it may interfere with the binding between Aha1 and Hsp90. Examples of such analogues include chemical compounds which are modelled to resemble the three dimensional structure of Hsp90 or Aha1 in the contact area, and in particular the arrangement of the key amino acid residues as they appear in Hsp90 or Aha1.

Hsp90 polypeptides and Aha1 polypeptides may be used in methods of designing mimetics of these molecules suitable for inhibiting Hsp90 activity.

Accordingly, the present invention provides a method of designing mimetics of Hsp90 and Aha1 polypeptides having the biological activity of activity of modulating, e.g. inhibiting, the Hsp90/Aha1 complex interaction or the activity of Hsp90, said method comprising:
(i) analysing a substance having the biological activity to determine the amino acid residues essential and important for the activity to define a pharmacophore; and,
(ii) modelling the pharmacophore to design and/or screen candidate mimetics having the biological activity.

Suitable modelling techniques are known in the art. This includes the design of so-called “mimetics” which involves the study of the functional interactions of the molecules and the design of compounds which contain functional groups arranged in such a manner that they could reproduced those interactions.

The modelling and modification of a ‘lead’ compound to optimise its properties, including the production of mimetics, is further described below.

An agent identified using one or more primary screens (e.g. in a cell-free system) as having ability to modulate (e.g. inhibit) the ATPase activity of Hsp90 may be assessed further using one or more secondary screens. A secondary screen may involve testing for a biological function of Hsp90 as noted above.

Suitable biological functions which may be assessed in a secondary screen include cellular proliferation, angiogenesis, apoptosis or migration/motility.

Determination of whether or not a test compound regulates a function such as cell proliferation may be performed for any particular cell line using conventional methods. For example, methods which may conveniently be used to assess the activity offered by a particular compound are described below.

For example, a sample of cells (e.g. from a tumour) may be grown in vitro and a test compound identified in a primary assay brought into contact with the cells. The effect of the compound on those cells is then observed. As examples of “effect,” the morphological status of the cells may be determined (e.g., alive or dead), the expression levels of genes associated with cell cycle regulation determined or effects on other aspects of cell function such as apoptosis or migration/motility may be determined. Growth inhibition assays also have application in the evaluation of candidate Hsp90 inhibitors. Further details of such assays are provided below.

Where the test compound is found to exert an influence on the cells, this may be used as a prognostic or diagnostic marker of the efficacy of the compound in methods of treating cells of the same type in a patient (e.g. a tumour or a tumour of the same cellular type).

In other secondary screens, molecular markers indicative of HSP90 inhibition (see, e.g. Whitesell, L. (1994) Proc. Natl. Acad. Sci. USA., Vol. 91, pp. 8324-8328; Clarke, P. A. et al (2000) Oncogene, Vol. 19, pp. 4125-33.) may be measured using Western blotting techniques or cell-based ELISA (enzyme-linked immunosorbent assay) (see, e.g., Stockwell et al. (1999) Chem. Biol., Vol. 6, pp. 71-83; Versteeg et al. (2000) Biochem. J., Vol. 350, pp. 717-722).

Assay methods of the present invention may therefore include determining the ability of said test compound to inhibit cellular proliferation.

As mentioned above, secondary screening may be performed to confirm the modulatory effect on Hsp90 activity of a compound identified by an assay method described herein. The cellular effects of Hsp90 inhibitors can be measured using a number of molecular markers. As already mentioned, HSP90 inhibition leads to the depletion of several important cellular signalling proteins. RAF-1 is readily detectable by Western blotting and has been shown to be depleted in a number of human tumour cell lines following exposure to HSP90 inhibitors (see, e.g., Kelland et al., 1999 supra; Hostein et al., (2001) Cancer Research Vol 61 pp. 4003-4009; Schulte et al., (1998) Cancer Chemother. Pharmacol. Vol. 42, pp. 273-279; Clarke et al., 2000 supra). Depletion is normally observed by 6 h, with maximum depletion occurring at 24 h. As well as RAF-1, depletion of several other HSP90 client proteins can be measured by immunoblotting e.g. CDK4, ErbB2. However, it is important to note that some of these proteins are cell line specific e.g. ErbB2 is expressed mainly in breast, thyroid, kidney and some ovarian tumour cell lines. Another very important marker of HSP90 inhibition is heat shock protein 70 (HSP70). A HSF-1 (heat shock factor 1) dependent increase in HSP70 levels has been reported (see, e.g., Whitesell et al. (1994) supra; Clarke et al. (2000) supra) and this effect can serve as a positive indicator of HSP90 inhibitor action. Immunoblotting methods to determine the presence or absence of these markers may be performed using standard techniques.

Western blotting has become a universally used technique for evaluating the level of protein expression in cell lines and tissue lysates and may be used for performing assay methods as described herein. Suitable protocols are well known in the art.

Western blotting has a number of potential drawbacks. The number of samples that can be included on each gel is limited and relatively large numbers of cells are required to detect proteins that are expressed at a low level. Also, precise quantitation is difficult.

Cell-based ELISA methods (see, e.g., Stockwell et al., 1999 supra; Versteeg et al. (2000) supra) offer several advantages for evaluating the pharmacodynamic effects of novel mechanism-based inhibitors and may be the method of choice for comparing inhibitors that are identified during the iterative process of lead identification and optimisation. The technique can be used to rapidly rank the effectiveness of compounds as well as to investigate the molecular mechanisms of their action. The increased sample throughput possible with ELISA means that compounds can be simultaneously studied in multiple replicates at different doses and exposure times. Also, the number of cells required per observation can be greatly reduced compared to those required for immunoblotting. The assays are carried out directly on cells grown and treated in microtitre plates. Alternatively, treated cells may be lysed directly in the wells of a microtitre plate, prior to ELISA on a separate plate. ELISA techniques may be applied to any cellular protein or post-translational modification for which an antibody is available and results are at least semi-quantitative.

A suitable growth inhibition assay may be based on known methods (Kelland et al. (1993) Cancer Research Vol. 53, pp. 2581-2586). Briefly, HCT116 and HT29 human colon tumour cells (American Tissue Culture Collection) may be seeded into 96-well tissue culture plates (approximately 1600-2000 cells per well) and allowed to attach for 36 hours. Eight wells may be treated with a single concentration from a range of compound concentrations and incubated for 96 hours. Cells may be fixed using ice cold 10% trichloroacetic acid (TCA). Plates may then be washed five times with water, air dried, and stained with 0.4% sulphorhodamine B (SRB) in 1% acetic acid for 10 minutes. The SRB stain may be solubilised in 10 mM Tris-HCl and the absorbance measured at 540 nm using a Titertek Multiscan MCC/340 MKII plate reader (Flow laboratories, IEC, Basingstoke, Hampshire). The absorbance values correspond to total protein content and may be used as a measure of cell growth. These values may be plotted on log/linear graph paper and the IC₅₀ was calculated as the drug concentration that inhibits cell growth by 50% compared with control cell growth.

Performance of an assay method according to the present invention may be followed by isolation and/or manufacture and/or use of a test compound which tests positive for ability to modulate interaction between Aha1 and Hsp90 and/or modulates (i.e. increases, enhances, reduces or inhibits) the Aha1-mediated ATPase activity of Hsp90.

Following identification of a test compound as an agent which has modulating activity, the test compound may be purified and/or isolated and/or investigated further.

It may be manufactured and/or used in preparation, i.e. manufacture or formulation, of a composition such as a medicament, pharmaceutical composition or drug, which may is be administered to individuals.

A method of producing a pharmaceutical composition, for example for use in the treatment of a disorder associated with cellular proliferation may comprise;
identifying a compound which modulates the activity of an Hsp90 polypeptide using a method described above; and,
admixing the compound identified thereby with a pharmaceutically acceptable carrier.

Pharmaceutically acceptable carriers are discussed in more detail below.

A method may comprise the step of modifying the compound to optimise the pharmaceutical properties thereof.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the optimisation of the lead compound.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

A method for preparing a pharmaceutical composition for treating a disorder of cellular proliferation may comprise;
i) identifying a compound which modulates the interaction of an AhaI polypeptide and an Hsp90 polypeptide,
ii) synthesising the identified compound, and;
iii) incorporating the compound into a pharmaceutical composition.

The identified compound may be synthesised using conventional chemical synthesis methodologies. Methods for the development and optimisation of synthetic routes are well known to a skilled person.

A compound found to have the ability to affect Hsp90 activity in an assay as described above has therapeutic and other potential in a number of contexts, as discussed, in particular for the treatment of disorders mediated by Hsp90 activity. For therapeutic treatment such a compound may be used in combination with any other active substance, e.g. for anti-tumour therapy, with another anti-tumour compound or therapy, such as radiotherapy or chemotherapy.

A test compound which is identified as a modulating agent may be used to obtain peptidyl or non-peptidyl mimetics, e.g. by methods well known to those skilled in the art and discussed herein. It may be used in a therapeutic context as discussed below.

Another aspect of the present invention provides an agent obtained or identified by an assay method described herein.

An agent identified by any one of the methods provided by the present invention may be isolated and/or purified and/or further investigated and/or manufactured. Various methods and uses of such compounds are discussed elsewhere herein.

An agent may be used in a method of regulating cell proliferation or other cellular properties of a malignant cell e.g. apoptosis, cell cycle, angiogenesis or metastasis. Such methods may comprise contacting a cell with an effective amount of an active compound, preferably in the form of a pharmaceutically acceptable composition. Such a method may be practised, for example, in vitro or in vivo.

As described above, one class of putative modulator compounds may be based on the Aha1 polypeptide sequence. Peptide fragments or alleles, mutants or derivatives of such fragments are described herein. Nucleic acid encoding s such peptides, vectors and host cells containing such nucleic acid, and methods of expressing nucleic acid encoding such peptides are further aspects of the present invention.

A suitable peptide fragment of Aha1 is able to interact with Hsp90 and/or inhibit interaction between Aha1 and Hsp90. Such a fragment may be used to modulate the ATPase activity of Hsp90.

Another aspect of the invention provides an Aha1 polypeptide as described herein which binds an Hsp90 polypeptide and/or stimulates the geldanamycin-sensitive ATPase activity of an Hsp90 polypeptide. The present invention also encompasses nucleic acid encoding an Aha1 polypeptide, which may be operably linked to a heterologous regulatory element and/or comprised within a vector.

Another aspect of the present invention provides a method of producing an Aha1 polypeptide comprising;
expressing said polypeptide from encoding nucleic acid; and,
determining the ability of said polypeptide to enhance the ATPase activity of Hsp90.

The invention further provides various therapeutic methods and uses of one or more substances selected from (i) an Aha1 polypeptide or fragment; (ii) a modulator identified by a screening method of the present invention; (iii) a mimetic of any of the above substances which modulates the Aha1 mediated ATPase activity of Hsp90.

Such a method or use may modulate, e.g. inhibit, reduce, enhance or increase the Hsp90 activity which is mediated by virtue of the interaction of Hsp90 with the co-factor Aha1.

The therapeutic/prophylactic purpose may be the treatment of a condition mediated by Hsp90.

The term “a condition mediated by Hsp90,” as used herein pertains to a condition in which Hsp90 and/or the action of Hsp90 is important or necessary, e.g., for the onset, progress, expression, etc. of that condition. Examples of conditions mediated by Hsp90 include, but are not limited to, a condition characterised by Hsp90 action upon a client protein which drives that condition; a condition characterised by one or more client proteins which are acted upon by Hsp90; a condition driven by one or more proteins, which proteins are Hsp90 client proteins, and which proteins could not drive the condition in the absence of action (e.g., chaperoning) by Hsp90; a condition driven by one or more proteins, which proteins are Hsp90 client proteins, and the action (e.g., chaperoning) by Hsp90 in order to drive the condition.

Examples of such conditions include, but are not limited to: cancer; immunosuppressive applications such auto-immune disease; arthritis; prion diseases (e.g., Creutzfeld Jacob Disease (CJD), variant CJD); other diseases associated with defects in protein folding and aggregation (e.g., Alzheimer's disease, Huntingdon's disease).

For example, many oncoproteins are HSP90 client proteins. In the absence of the chaperoning action of HSP90, these proteins are degraded, for example, by ubiquitin dependent proteasome degradation. Similarly, LCK protein, characteristic of many autoimmune diseases, is also an HSP90 client protein. In the absence of the chaperoning action of HSP90, LCK levels are reduced.

In one embodiment, the present invention provides methods for obtaining anticancer agents. The term “anticancer agent” as used herein, pertains to a compound which treats a cancer (i.e., a compound which is useful in the treatment of a cancer). The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

One of ordinary skill in the art is readily able to determine whether or not a candidate compound treats a cancerous condition for any particular cell type. For example, methods which may conveniently be used to assess the activity offered by a particular compound are described herein.

The present invention also provides active compounds which are antiproliferative agents. The term “antiproliferative agent” as used herein, pertains to a compound which treats a proliferative condition (i.e. a compound which is useful in the treatment of a proliferative condition).

One of ordinary skill in the art is readily able to determine whether or not a candidate compound treats a proliferative condition for any particular cell type. For example, methods which may conveniently be used to assess the activity offered by a particular compound are described in the examples below.

The terms “cell proliferation,” “proliferative condition,” “proliferative disorder,” and “proliferative disease,” are used interchangeably herein and pertain to an unwanted or uncontrolled cellular proliferation of excessive or abnormal cells which is undesired, such as, neoplastic or hyperplastic growth, whether in vitro or in vivo.

A proliferative disorder is an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, (e.g., histocytoma, glioma, astrocyoma, osteoma), cancers (e.g., lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), leukemias, psoriasis, bone diseases, fibroproliferative disorders (e.g., of connective tissues), cataracts and atherosclerosis.

A proliferative disorder may occur in any cell-type, including but not limited to, lung, colon, breast, ovarian, prostate, liver, pancreas, brain, and skin.

A compound obtained by the present methods may be either one or both of an ‘anti-cancer agent’ and an ‘anti-proliferative agent’.

As described above, an agent may be used for cancer treatment, which may, for example, be in combination with chemotherapy and/or radiotherapy, or cancer prophylaxis. The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

In various further aspects, the present invention thus provides a pharmaceutical composition, medicament, drug or other composition for such a purpose comprising one or more compounds or agents as described herein, the use of such a composition in a method of medical treatment, a method comprising administration of such a composition to an individual or patient, e.g. for treatment (which may include preventative treatment) of a medical condition, e.g. a condition mediated by Hsp90, such as a condition associated with a defect or disorder in transcriptional control, DNA replication, or cell cycle control, such as for treatment of cancer or other disorder of cellular proliferation, use of a compound or agent as described herein in the manufacture of a composition, medicament or drug for administration for such a purpose, e.g. for treatment of a disorder mediated by Hsp90, a compound agent or composition for use in treating a condition mediated by Hsp90, and a method of making a pharmaceutical composition comprising admixing such a substance with a pharmaceutically acceptable excipient, vehicle or carrier, and optionally other ingredients.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

Whatever the compound or agent used in a method of medical treatment of the present invention, administration is preferably in a ‘prophylactically effective amount’ or a ‘therapeutically effective amount’ as the case may be (although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

The term “therapeutically-effective amount”, as used herein, pertains to that amount of an active compound, or a material, composition or dosage from comprising an active compound, which is effective for producing some desired therapeutic effect, commensurate with a reasonable benefit/risk ratio.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect.

Administration in vivo can be effected in one dose, continuously or intermittently throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Compounds identified as agents with modulating activity may also be used as cell culture additives to inhibit HSP90, for example, in order to regulate cell proliferation, apoptosis, cell cycle or angiogenesis/migration in vitro. Such compounds may also be used as part of an in vitro assay, for example, in order to determine whether a candidate host is likely to benefit from treatment with the compound in question. They may also be used as a standard, for example, in an assay, in order to identify other active compounds, other HSP90 inhibitors, other anti-proliferative agents, etc.

A substance or composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient.

Thus, the present invention further provides pharmaceutical compositions, as defined above, and methods of making a pharmaceutical composition comprising admixing at least one active ingredient, as defined above, together with one or more pharmaceutically acceptable carriers, excipients, buffers, adjuvants, stabilisers, or other materials, as described herein.

The term “pharmaceutically acceptable” pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be oral, or by injection, e.g. cutaneous, subcutaneous or intravenous.

Formulations may conveniently be presented in unit dosage form and may be prepared by any methods well known in the art of pharmacy. Such methods include the step of bringing into association the active ingredient with the carrier, which constitutes one or more accessory ingredients. In general, the formulations are prepared by uniformly and intimately bringing into association the active ingredient with liquid carriers or finely divided solid carriers or both, and then if necessary shaping the product.

Formulations may be in the form of liquids, solutions, suspensions, emulsions, tablets, losenges, granules, powders, capsules, cachets, pills, ampoules, suppositories, pessaries, ointments, gels, pastes, creams, sprays, foams, lotions, oils, boluses, electuaries, or aerosols.

The active compound or pharmaceutical composition comprising the active compound may be administered to a subject by any convenient route of administration, whether systemically/ peripherally or topically (i.e., at the site of desired action).

Routes of administration include, but are not limited to, oral (e.g. by ingestion); buccal; sublingual; transdermal (including, e.g., by a patch, plaster, etc.); transmucosal (e.g. by a patch, plaster, etc.); intranasal (e.g. by nasal spray); ocular (e.g. by eyedrops); pulmonary (e.g. by inhalation or insufflation therapy, for example via an aerosol through the mouth or nose); rectal (e.g. by suppository or enema); vaginal (e.g. by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose), surface-active or dispersing agent. Moulded tablets may be made by moulding, in a suitable machine, a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Liposomes, particularly cationic liposomes, may be used in carrier formulations.

Examples of techniques and protocols mentioned above can be found in Remington's Pharmaceutical Sciences, 16th edition, Osol, A. (ed), 1980.

Another aspect of the present invention provides a method of making a pharmaceutical composition comprising admixing an agent identified by an assay as described above with a pharmaceutically acceptable excipient, vehicle or carrier.

The composition may be administered in a localised manner to a tumour site or other desired site or may be delivered in a manner in which it targets tumour or other cells.

Targeting therapies may be used to deliver the active compound or agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering such agents directly, they may be produced in the target cells by expression from an encoding nucleic acid introduced into the cells, e.g. from a viral vector. The vector may be targeted to the specific cells to be treated, or it may contain regulatory elements which are switched on more or less selectively by the target cells.

Nucleic acid encoding the agent e.g. a peptide able to modulate, e.g. interfere with, the interaction of Aha1 and Hsp90 and thereby affect Hsp90 activity may thus be used in methods of gene therapy, for instance in treatment of individuals, e.g. with the aim of preventing or curing (wholly or partially) a disorder.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

A peptide or other compound having the ability to modulate or interfere with the interaction of Aha1 and Hsp90 and/or the Aha1 mediated ATPase activity of HBP90, or a nucleic acid molecule which encodes a peptide having that ability, may be provided in a kit, e.g. sealed in a suitable container which protects its contents from the external environment. Such a kit may include instructions for use.

Various further aspects and embodiments of the present invention will be apparent to those skilled in the art in view of the present disclosure.

Certain aspects and embodiments of the invention will now be illustrated by way of example and with reference to the figures described below.

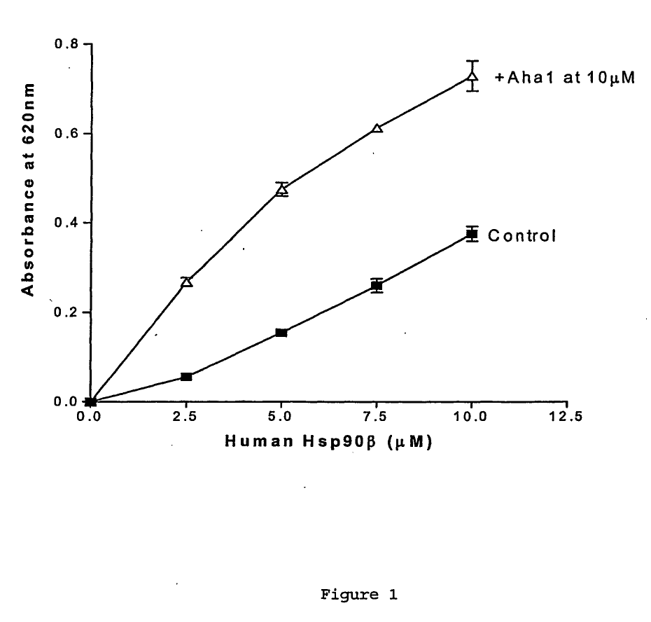

FIG. 1 shows the effect of the co-activator, Aha1 on the ATPase activity of increasing concentrations of human HSP90. Each value is the mean±Sd of n=4 ATP at 0.6 mM.

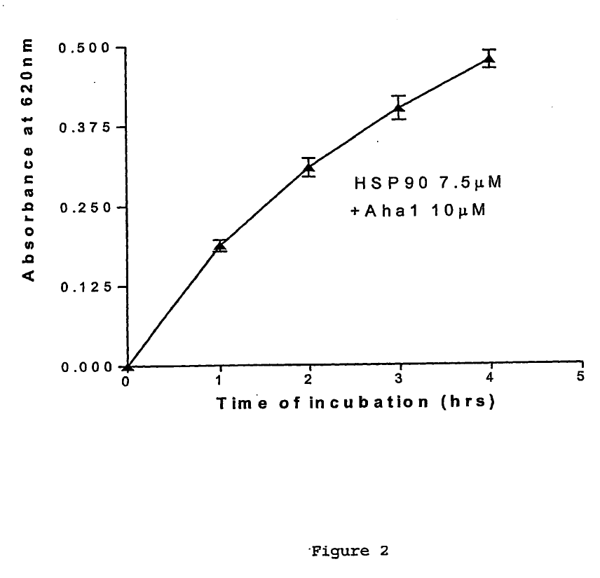

FIG. 2 demonstrates the relationship between ATPase activity and time for human HSP90 in the presence of Aha1. Each point is the mean±SD of n=4 ATP at 0.6 mM.

FIG. 3 shows an alignment of Aha1 polypeptides from different species: Sc Saccharomyces cerevisiae, Ca Candida albicans, At Arabidopsis thaliana, Dm Drosophila melanogaster, Ce Caenorhabditis elegans, Hs Homo sapiens, Sp Schizosaccharomyces pombe.

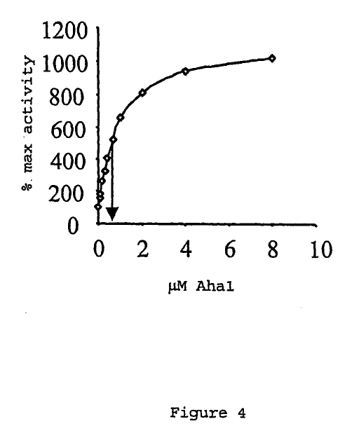

FIG. 4 shows the effect of Aha1 concentration on the geldanamycin dependent ATPase activity of Hsp90. Half activation is shown to be at <1 mM Aha1. 0.2 μM Aha1 was used in a buffer consisting of 45 mM Tris pH 7.5, 9 mM KCl, 3 mM MgCl₂.

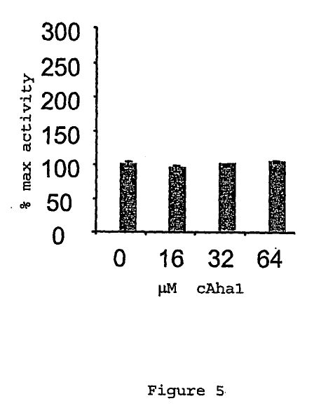

FIG. 5 shows the effect of increasing concentrations of the C terminal domain of Aha1 on geldanamycin dependent ATPase activity of Hsp90. 2 μM Aha1 was used in a buffer consisting of 20 mM Tris pH 7.5, 4 mM KCl, 1.2 mM MgCl₂.

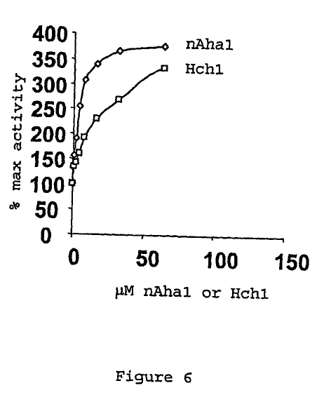

FIG. 6 shows the effect of increasing concentrations of Hch1 or the N terminal domain of Aha1 on geldanamycin dependent ATPase activity of Hsp90. 2 μM Aha1 was used in a buffer consisting of 20 mM Tris pH 7.5, 4 mM KCl, 1.2 mM MgCl₂.

Table 1 shows an non-exclusive list of known HSP90 proteins (from: Gupta (1995) Mol Biol Evol 12 (6) 1063-1073). SP is SwissProt, EM is EMBL and GB is Genbank.

Table 2 shows the results of IC₅₀ experiments for radicicol, geldanamycin and 17AAG using yeast and human Hsp90 in the presence and absence of Aha1. IC50±SD values are shown for >3 determinations.

### EXPERIMENTAL

Material and Methods

Expression of Proteins

The Aha1 (YDR214W) gene was cloned into pRSETA a vector carrying a T7 promoter. The protein was produced by expressing the gene from the pRSETA vector in E. coli BL21 (DE3) pLysS and by inducing with 1 mM IPTG. Cells were harvested and lysed in 20 mM Tris pH 8.0 containing 100 mM NaCl (Buffer A) and protease inhibitors (Boehringer Protease Inhibitor Tablets without EDTA) and centrifuged.

The lysate was then applied to a Talon metal affinity column and washed with the same buffer, followed by a wash with Buffer A containing 10 mM imidazole at pH 8.0. The protein was eluted with Buffer A containing 300 mM imidazole at pH 7.6. 10 mM EDTA and 1 mM DTT was added to the eluted protein which was then concentrated and applied to a Superdex 200 PG column equilibrated in 20 mM Tris pH 7.4 containing 1 mM EDTA and 500 mM NaCl.

The fractions containing Aha1 were then dialysed (20 mM Tris 7.4, 1 mM EDTA) and applied to a Q-sepharose column equilibrated in 20 mM Tris 7.4, 1 mM EDTA. The bound protein was eluted with a NaCl gradient of 0 to 1 M in the same equilibration buffer and fractions containing Aha1 dialysed against 20 mM Tris 7.4 and 1 mM EDTA. The protein was then concentrated using Vivaspin SK concentrators.

The purification of the Human hsp90 beta protein is almost identical to that of the Aha1 protein—except that the gel filtration is carried out with a Sephacryl 400 HR column and concentrated in a Vivaspin 30K concentrator. The buffers used are identical.

Hch1 and truncated Aha1 polypeptides corresponding to the N and C terminal domains of full-length Aha1 were expressed and purified as described above. The N terminal domain polypeptide (nAha1) consisted of 162 aa from the N terminal MVVNNP to GNDIQV. The C terminal domain polypeptide (cAha1) consisted of the remainder of the Aha1 sequence.

Colorimetric Assay to Determine ATPase Activity of Hsp90.

The activity of enzymes that release inorganic phosphate can be measured using the reaction of the cationic dye, malachite green, with a phosphomolybdate complex to generate a blue-green colour with an absorbance maximum at 610 nm (Cogan, E. B. et al (1999) Anal. Biochem. 271, 29-35; Baykov, A. A. et al (1988) Anal. Biochem. 171, 266-270; Harder, K. W. et al (1994) Biochem. J. 298, 395-401; Maehama, T. et al (2000) Anal. Biochem. 279, 248-250) as described above.

Assay Reagents

Chemicals are of the highest purity commercially available and all aqueous solutions are made up in AR water.
1. For the full length Aha1, an assay buffer of 45 mM Tris-HCl pH 7.5, 9 mM KCl, 3 mM MgCl may be used. For the C-and N-terminal domain of Aha1 or the Hch1 homologue, an 20 mM Tris-HCl pH 7.5, 4 mM KCl, 1.2 mM MgCl. An assay buffer of 100 mM Tris-HCl, pH 7.4, 20 mM KCl, 6 mM MgCl₂ may also be used. Stored at 4° C.
2. 0.0812% (w/v) malachite green (Sigma M 9636). Stored at room temperature.
3. 2.32% (w/v) polyvinyl alcohol USP (Sigma P 1097). Stored at room temperature. The polyvinyl alcohol dissolves in boiling water with difficulty and stirring for 2-3 h is required
4. 5.72% (w/v) ammonium molybdate in 6M hydrochloric acid. Stored at room temperature.
5. 34% (w/v) sodium citrate. Stored at room temperature.
6. ATP, disodium salt, special quality (Boehringer Mannheim 519979). Stored at 4° C.
7. E. coli expressed yeast HSP90 protein, purified >95% (10) and stored at −80° C. as 10 μl aliquots containing 0.5 mg of protein.
   Assay Protocol

On the day of use, the malachite green reagent was prepared from the stock solutions; 2 parts of malachite green were mixed with 1 part each of polyvinyl alcohol and ammonium molybdate and 2 parts of water.

Initially, the reagent was a dark brown colour, but after standing at room temperature for about 2 h, this became a golden yellow colour and was ready for use. All assays were run in Immulon 96-well flat-bottomed clear polystyrene plates.

ATP was dissolved in the assay buffer to give a stock concentration of 1.5 mM and stored at room temperature. A 10 μl aliquot of ATP solution was added to each well to give a final assay concentration of 0.6 mM. Immediately before use, Aha 1 and human HSP90 protein were thawed on ice and suspended in chilled assay buffer to stock concentrations of 50 μM (2.03 μg/μl) and 25 μM (2.11 μg/μl) respectively and the solutions kept on ice.

Aha1 (5 μl) was added to each well giving a final concentration of 10 μM, 5 μl buffer was added to wells not containing Aha1. The incubation was started by the addition of 7.5 μl of stock HSP90 to each well (except for the background wells which received 7.5 μl of assay buffer) giving a final assay volume of 25 μl. The plates were shaken (approximately 2 min) using a plate shaker (e.g. Wellmixx (Thermo Labsystems) or MTS4 (IKA-Schuttler)), sealed with plastic film and incubated for 3 h at 37° C.

To stop the incubation, 80 μl of the malachite green reagent was added to each well and the plate shaken again. 10 μl of 34% sodium citrate was added to each well and the plate shaken again. The absorbance at 620 nm was measured using a suitable plate reader (e.g. Victor², PerkinElmer Life Sciences).

For IC50 determinations of Hsp90 inhibitors, a range of stock concentrations of the compound in DMSO was prepared. Four appropriate concentrations were used depending on the relative potency of each compound. A 1 μl aliquot of each concentration was transferred to the wells of the assay plate and the assay carried out as described above. The time interval between addition of the malachite green reagent and the sodium citrate should be kept as short as possible in order to reduce the non-enzymatic hydrolysis of ATP. Once the sodium citrate is added, the colour is stable for up to 4 h at room temperature.

Results

The assay conditions were optimised with respect to time and protein at a substrate concentration of 0.6 mM ATP in order to achieve linearity of enzyme activity under the described protocol.

The presence of the co-activator, Aha1, was observed to significantly increase the ATPase activity of increasing concentrations of human HSP90 (FIG. 1).

The increase in ATPase activity in the presence of Aha1 continued to develop for at least several hours (FIG. 2).

Half maximal activation of Hsp90 was found to occur at concentrations of less than 1 μM Aha1 (arrow; FIG. 4).

The C terminal domain of Aha1 was shown to have little or no effect on Hsp90 activity (FIG. 5) but the N terminal domain of Aha1 (FIG. 6 -⋄-) and also the homologue Hch1 (FIG. 6 -□-) were both shown to activate Hsp90 ATPase activity, but to a lesser extent than the full length Aha1 polypeptide (FIG. 6).

IC50 Determinations

For the IC50 determinations, all assays were run with 7.5 μM human HSP90 and 10 μM Aha1.

The yeast assay was run using 1.6 μM protein and ATP at 1 mM. The human protein was assayed as described above. In all cases, the assay volume was 25 μl and the plates were incubated for 3 hrs at 37° C. The results are shown as IC50 values in Table 2, the units are μM and ± represents SD of n=3-5, where available.

The three compounds used (radicicol, geldanamycin and 17AAG) are potent inhibitors of the yeast HSP90 ATPase activity. At the concentrations used, they were not observed to inhibit the basal activity observed with the human protein alone. In other words, in the absence of Aha1, ATPase activity measured is insensitive to inhibition by geldanamycin or other known yeast HSP90 ATPase inhibitors.

This basal geldanamycin-insensitive activity may be due to human Hsp90 or a contaminating ATPase.

The activity of human Hsp90 is increased approximately 2-fold in the presence of Aha1. In addition the increased activity is sensitive to geldanamycin. The inhibitory potency of radicicol, geldanamycin and 17AAG against the human HSP90 ATPase in the presence of the co-activator, Aha1, was similar to that shown against the yeast enzyme.

Inhibition of yeast Hsp90 ATPase activity using known inhibitors such as radicicol, geldanamycin or 17-AAG, has been shown to prevent recruitment of co-chaperones and encourage the formation of a type of Hsp90 heterocomplex from which these client proteins are targeted for degradation via the ubiquitin proteosome pathway (see, e.g., Neckers L et al (1999) Invest. New Drugs, Vol. 17, pp. 361-373; Kelland et al. (1999) J. Natl. Cancer Inst. Vol. 91, pp. 1940-1949). Treatment with Hsp90 inhibitors may therefore lead to the selective degradation of important proteins involved in cell proliferation, cell cycle regulation and apoptosis. All these processes are fundamentally important in conditions such as cancer.

The methods described herein for determining the activity of human Hsp90 in the presence of the co-activator Aha1 are therefore useful in the development of novel inhibitors of Hsp90 for clinical applications.

TABLE 1
Hsp90 Polypeptides

| Name | Accession Number |
|---|---|
| Human α | SP/Po7900 |
| Mouse α | SP/P07901 |
| Chicken α | SP/P11501 |
| Human β | SP/P08238 |
| Mouse β | GB/M36829 |
| Rat β | GB/S45392 |
| Chicken β | SP/Q04619 |
| Drosophila melanogaster | SP/P02828 |
| Maize | GB/S59780 |
| Arabidopsis thaliana | SP/P27323 |
| Pharbitis nil | GB/M99431 |
| Rice | SP/P33126 |
| Tomato | GB/M96549 |
| Trypanosoma brucei | SP/P12861 |
| Trypanosoma cruzi | SP/P06660 |
| Leishmania amazonensis | SP/P27741 |
| Plasmodium falciparum | GB/L34027 |
| Theileria parva | SP/P24724 |
| S. cerevisiae | SP/P15108 |
| S. cerevisiae | SP/P02829 |
| Ajellomyces capsulata | GB/S21764 |
| Histoplasma capsulatum | GB/M55629 |
| Human (ER homol) | SP/P24625 |
| Mouse (ER homol) | SP/P08113 |
| Dog (ER homol) | GB/U01153 |
| Pig (ER homol) | GB/X76301 |
| Chicken (ER homol) | SP/P08110 |
| Barley (ER homol) | EM/S31862 |
| C. roseus (ER homol) | GB/L14594 |
| Secale cereale(ER homol) | GB/Z30243 |
| E. coli (ER homol) | SP/P10413 |

TABLE 2
Inhibition (IC50) of Hsp90 ATPase activity (μM)

| | | | Human |
|---|---|---|---|
| Compounds | Yeast Hsp90 | Human Hsp90 | Hsp90 + Aha1 |
| Radicicol | 0.9 ± 0.4 | >10 | 2.2/2.9 |
| 17AAG | 8.7 ± 2.3 | >80 | 3.7/3.4 |
| Geldanamycin | 4.8 ± 0.8 | >80 | ˜5 |

1. An method for obtaining an agent which modulates the interaction between Aha1 and Hsp90 including;
(a) bringing into contact an Hsp90 polypeptide, a Aha1 polypeptide and a test compound; and,
(b) determining interaction between said Aha1 polypeptide and said Hsp90 polypeptide.

2. A method according to claim 1 comprising the step of determining ATPase activity of said Hsp90 polypeptide.

3. A method for obtaining an agent which modulates ATPase activity of Hsp90, the method comprising:
(a) bringing into contact an Hsp90 polypeptide, an Aha1 polypeptide and a test compound; and,
(b) determining ATPase activity of said Hsp90 polypeptide.

4. A method according to claim 1 comprising determining the production of inorganic phosphate by said Hsp90 polypeptide.

5. A method according to claim 4 wherein said production of inorganic phosphate is determined by determining the production of a reporter molecule.

6. A method according to claim 5 wherein said reporter molecule is produced by the reaction of a cationic dye with a phosphomolybdate complex.

7. A method according to claim 1 wherein the Hsp90 polypeptide is a human Hsp90 polypeptide.

8. A method according to claim 7 wherein the Hsp90 polypeptide is selected from the group consisting of Hsp90α, Hsp90β, GRP94 and Hsp75/TRAP1.

9. A method according to claim 8 wherein the Hsp90 polypeptide has the sequence of database entry EMBL:SCHSP90 K01387.

10. A method according to claim 1 wherein the Aha1 polypeptide is an Aha1 polypeptide shown in FIG. 3.

11. A method according to claim 10 wherein the Aha1 polypeptide is a yeast polypeptide having the sequence of Genbank Accession number YDR214W.

12. A method according to claim 10 wherein the Aha1 polypeptide is a human polypeptide having the sequence of Genbank Accession number AJ243310.

13. A method according to claim 1 including determining the ability of said test compound to modulate cellular levels of one or more marker proteins.

14. A method according to claim 13 wherein the one or more marker proteins are selected from the group consisting of RAF-1, CDK4, ErbB2 and Hsp70.

15. A method according to claim 1 including determining the ability of said test compound to inhibit one or more of cell growth, cell motility, cell proliferation, apoptosis, cell cycle events, angiogenesis and metastasis.

16. A method according to claim 1 including identifying said test compound as an agent which modulates the activity of Hsp90.

17. A method according to claim 16 comprising isolating and/or purifying said test compound.

18. A method according to claim 16 comprising formulating said agent into a composition which includes one or more additional components.

19. A method according to claim 18 wherein said one or more additional components include a pharmaceutically acceptable excipient.

20. A method of producing a pharmaceutical composition comprising;
identifying a compound which modulates the activity of an Hsp90 polypeptide using a method according to claim 1; and,
admixing the compound identified thereby with a pharmaceutically acceptable carrier.

21. A method according to claim 20 comprising the step of modifying the compound to optimise the pharmaceutical properties thereof.

22. A method for preparing a pharmaceutical composition for treating a disorder of cellular proliferation,
comprising;
i) identifying a compound which modulates the interaction of an Aha1 polypeptide and an Hsp90 polypeptide,
ii) synthesising the identified compound, and;
iii) incorporating the compound into a pharmaceutical composition.

23. An agent obtained by an assay method according to claim 1.

24. A pharmaceutical composition comprising an agent according to claim 23.

25. A method of treatment of a disorder mediated by Hsp90 comprising administering a composition according to claim 24.

26. A method according to claim 25 wherein the composition comprises an Aha1 polypeptide.

27. A method of producing an Aha1 polypeptide comprising;
expressing said polypeptide from encoding nucleic acid; and,
determining the ability of said polypeptide to enhance the ATPase activity of Hsp90.

28. A method of making a pharmaceutical composition comprising admixing an agent according to claim 23 with a pharmaceutically acceptable excipient, vehicle or carrier.

29. A method comprising administration of a composition according to claim 24 to an individual for treatment of a disorder of cellular proliferation.
